(19) United States
(12) Patent Application Publication
(10) Pub. No.: US 2013/0253884 A1
(43) Pub. Date: Sep. 26, 2013

(54) METHOD AND APPARATUS FOR MORPHOLOGICAL ANALYSIS

(71) Applicants: Gustavo Aizenberg, Ness Ziona (IL); David Pincu, Holon (IL)

(72) Inventors: Gustavo Aizenberg, Ness Ziona (IL); David Pincu, Holon (IL)

(73) Assignee: G & D INNOVATIVE ANALYSIS, LTD., Ness Ziona (IL)

(21) Appl. No.: 13/990,087

(22) PCT Filed: Mar. 14, 2012
(86) PCT No.: PCT/IL2012/050091

§ 371 Date: May 29, 2013

Related U.S. Application Data
(60) Provisional application No. 61/452,287, filed on Mar. 14, 2011.

(51) Int. Cl. G01B 11/24; A61B 3/10; G01B 11/06

(52) U.S. Cl. CPC G01B 11/24; G01B 11/06; A61B 3/102
USPC 702/167; 356/601; 351/221

(57) ABSTRACT

An apparatus arranged to analyze a structure, the apparatus comprising: a control unit; a light source arranged to irradiate a target area of the structure; and a light detector in communication with said control unit, and arranged to detect said irradiated light from said light source after interaction with the target area, said control unit arranged to: transform amplitudes of the detected light to the optical thickness domain, said transform comprising a bilinear transform; determine morphological information of the target area responsive to said performed transform. The determined morphological information is optionally displayed within a three dimensional view of the target area.

### TECHNICAL FIELD

The present disclosure relates generally to the field of analyzing structures and in particular to a method and apparatus for analyzing morphological properties of a structure.

### BACKGROUND ART

The application of optical coherence tomography (OCT) has increased significantly over the past several years. OCT has been applied not only to ophthalmology, but also to other medical fields such as the investigation of skin diseases, endoscopy and colonoscopy. Moreover, it has been used for non-medical fields, such as evaluation of materials, for example in the evaluation of cracks in silicon nitride bearing balls and in the analysis of art objects.

Common OCT devices are based on Michelson interferometer principles. Specifically, a beam of light is split into two beams of light, where a first beam of light interacts with the sample and the second beam of light is reflected off a reference mirror to interfere with the first beam of light after the interaction of the first beam of light with the sample. The interference signal carries information about the layers forming the sample. The Michelson interferometer is very useful for optical analysis deep within the specimen, such as in the case of the human retina where it is typically positioned at about two centimeters from the human body surface.

Since some OCT applications, such as: the analysis of the cornea or skin; colonoscopy; analysis of materials; and the analysis of art objects, need to be analyzed at a relatively shallow depth, a simple and cheap analyzing tool arranged to provide optical analysis at shallow depths would be advantageous.

An article by G. E. Aizenberg, et al. entitled “A Digital Signal-Processing Analysis Technique for the Infrared Reflectivity Characterization of Ion Implanted Silicon”, published in Journal of Electronic Material, Vol. 21, No. 11, in 1992, describes a method for analyzing optical reflectance by performing a Fourier Transform of a Bilinear Transform of the reflectance data. An article by G. E. Aizenberg, et al. entitled “Optical characterization of semiconductors containing inhomogeneous layers”, published in Applied Surface Science, Vol. 63, in 1993, also describes the above analyzing method. However, the aforementioned articles do not teach or suggest how to build a practical apparatus for implementing the mathematical methods described therein.

### SUMMARY OF INVENTION

In view of the discussion provided above and other considerations, the present disclosure provides methods and apparatus to overcome some or all of the disadvantages of prior and present methods of providing analysis of structures. Other new and useful advantages of the present methods and apparatus will also be described herein and can be appreciated by those skilled in the art.

In certain embodiments an apparatus arranged to analyze a structure is provided, the apparatus comprising: a control unit; a target focusing functionality; a light source in optical communication with the target focusing functionality and arranged to irradiate a target area of the structure in cooperation with the target focusing functionality; and a light detector, in communication with the control unit and arranged to detect the irradiated light from the light source after interaction with the target area. The control unit is arranged, for each of a plurality of target areas of the structure, to: control the target focusing functionality such that the target area is irradiated by the light source and the light detector detects the irradiated light from the light source after interaction with the target area; detect the amplitude of the detected light as a function of wavelength; perform a transform of a function of the detected amplitudes to the optical thickness domain; determine morphological information of the target area responsive to the performed transform; and output the determined morphological information.

In one independent embodiment, an apparatus arranged to analyze a structure is provided, the apparatus comprising: a control unit; a light source arranged to irradiate a target area of the structure; and a light detector in communication with the control unit, and arranged to detect the irradiated light from the light source after interaction with the target area, the control unit arranged to: transform amplitudes of the detected light to the optical thickness domain, the transform comprising a bilinear transform; determine morphological information of the target area responsive to the performed transform; and output the determined morphological information.

In one embodiment, the apparatus further comprises: a target focusing functionality in optical communication with the light source, wherein the arrangement of the light source to irradiate a target area is in cooperation with the target focusing functionality, wherein the control unit is further arranged, for each of a plurality of target areas of the structure, to control the target focusing functionality such that the target area is irradiated by the light source and the light detector detects the irradiated light from the light source after interaction with the target area, and wherein the arrangement of the control unit to transform, determine and output is for each of the plurality of target areas. In one further embodiment, the apparatus further comprises: a display, in communication with the control unit and arranged to display the output morphological information, wherein the morphological information comprises refractive index information of a plurality of layers of each target area, and wherein a three dimensional view of the target area is displayed on the display, the determined refractive index information of each layer of each target area being displayed within the three dimensional view.

In one embodiment, the morphological information comprises optical thickness information of the target area. In another embodiment, the morphological information comprises the refractive index of one layer of the target area.

In one embodiment, the morphological information comprises the thickness of at least one layer of the target area. In another embodiment, the control unit is further arranged to receive topographic information of a surface of the target area and adjust the calculated morphological information responsive to the received topographic information.

In one embodiment, the structure is a Cornea and the morphological information comprises the thickness of the Cornea.

In another independent embodiment, a method of analysis of a structure is provided, the method comprising: irradiating a target area of the structure with a first beam of light; detecting the amplitude of the first beam of light after interaction with the target area, the amplitude detected as a function of wavelength; transforming the detected amplitudes to the optical thickness domain, the transforming comprising performing a bilinear transform on the detected amplitudes; determining morphological information of the target area responsive to the transforming; and outputting the determined morphological information.

In one embodiment, the irradiating, detecting, transforming, determining and outputting is performed for each of a plurality of target areas of the structure. In one further embodiment, the morphological information comprises refractive index information of a plurality of layers of each target area and the method further comprises: displaying a three dimensional view of the target area; and displaying the determined refractive index information of each layer of each target area within the displayed three dimensional view.

In one embodiment, the morphological information comprises optical thickness information of a layer of the target area. In another embodiment, the morphological information comprises the refractive index of a layer of the target area.

In one embodiment, the morphological information comprises the thickness of a layer of the target area. In another embodiment, the method further comprises disposing a layer of optical material, exhibiting a known refractive index, on the structure.

In one embodiment, the method further comprises: receiving topographic information of a surface of the target area; and adjusting the calculated morphological information responsive to the received topographic information. In another embodiment, the structure is a Cornea and the morphological information comprises the thickness of the Cornea.

In one independent embodiment, a control unit arranged to analyze light reflected off a target area of a structure is provided, the amplitudes of the light reflected off the target area detected by a detector arranged to output information regarding the detected amplitudes as a function of wavelength, the control unit comprising: a transform functionality arranged to transform the detected amplitudes to the optical thickness domain, the transform comprising a bilinear transform; and a determining functionality arranged to determine morphological information of the target area responsive to the transform and further arranged to output the determined morphological information.

In one embodiment, the control unit further comprises: an irradiating control functionality arranged, for each of a plurality of target areas of the structure, to control a target focusing functionality such that the target area is irradiated by light, wherein the arrangement to transform, determine and output is for each of the plurality of target areas. In one further embodiment, the control unit is in communication with a display arranged to display the output morphological information, wherein the morphological information comprises refractive index information of a plurality of layers of each target area, and wherein the control unit is arranged to cause the display to display a three dimensional view of the target area and to display the determined refractive index information of each layer of each target area within the three dimensional view.

In one embodiment, the morphological information comprises optical thickness information of a layer of the target area. In another embodiment, the morphological information comprises the refractive index of a layer of the target area.

In one embodiment, the morphological information comprises the thickness of a layer of the target area. In another embodiment, the determining functionality is further arranged to receive topographic information of a surface of the target area and adjust the calculated morphological information responsive to the received topographic information.

In another independent embodiment, a method of measuring the thickness of a target portion of a Cornea is provided, the method comprising: irradiating the target portion of the Cornea with a first beam of light; detecting the first beam of light after interaction with the target portion of the Cornea; determining the thickness of the target portion of the Cornea responsive to a reflectometric analysis of the detected light; and outputting the determined thickness of the target portion of the Cornea.

In one embodiment, the detecting comprises detecting the amplitude of the detected light as a function of wavelength, and the method further comprises: transforming the detected amplitudes to the optical thickness domain, the transforming comprising performing a bilinear transform on the detected amplitudes; and determining morphological information of the target area responsive to the performed transform, the determined morphological information comprising the determined thickness. In one further embodiment, the irradiating, detecting, transforming, determining morphological information and outputting is performed for each of a plurality of target areas of the cornea.

In one embodiment, the morphological information comprises optical thickness information of the cornea. In another embodiment, the morphological information comprises the refractive index of a layer of the cornea.

In one embodiment, the morphological information comprises the thickness of a layer of the cornea. In another embodiment, the method further comprises: receiving topographic information of a surface of the cornea; and adjusting the calculated morphological information responsive to the received topographic information.

In one embodiment, the morphological information comprises refractive index information of a plurality of layers of each target area, and the method further comprises: displaying a three dimensional view of the target area; and displaying the determined refractive index information of each layer of each target area within the displayed three dimensional view.

Additional features and advantages of the invention will become apparent from the following drawings and description.

### BRIEF DESCRIPTION OF DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1A illustrates a high level schematic diagram of an apparatus arranged to determine morphological information of a structure responsive to light reflected off the structure, according to certain embodiments;

FIG. 1B illustrates a high level block diagram of a processor of the apparatus of FIG. 1A, according to certain embodiments;

FIG. 1C illustrates a high level flow chart of the operation of the apparatus of FIG. 1A to determine morphological data of a target structure;

FIG. 1D illustrates a high level flow chart of the operation of the processor of FIG. 1B to determine morphological data;

FIG. 1E illustrates a high level diagram of a multi-layer structure;

FIG. 1F illustrates a plot of reflectance data vs. wave number of a light beam reflected from the structure of FIG. 1E;

FIG. 1G illustrates a plot of the reflectance data of FIG. 1E after performing a bilinear transform and a Fourier transform to the optical thickness domain;

FIG. 1H illustrates a high level flow chart of the operation of the apparatus of FIG. 1A to determine the thickness of a target Cornea;

FIG. 2A illustrates a high level schematic diagram of an apparatus arranged to determine morphological information of a structure responsive to light reflected off the structure and further responsive to topographic information of the surface of the structure, according to certain embodiments;

FIG. 2B illustrates a high level flow chart of the operation of the apparatus of FIG. 2A, according to certain embodiments;

FIG. 3A illustrates a high level schematic diagram of an apparatus arranged to determine morphological information of a structure responsive to interference between light reflected off the structure and a reference light, according to certain embodiments;

FIG. 3B illustrates a high level flow chart of the operation of the apparatus of FIG. 3A, according to certain embodiments;

FIG. 4A illustrates a high level schematic diagram of an apparatus arranged to determine morphological information of a multi-layer optical structure in a plurality of modes, according to certain embodiments; and

FIG. 4B illustrates a high level flow chart of the operation of the apparatus of FIG. 4A.

### DESCRIPTION OF EMBODIMENTS

Before explaining at least one embodiment in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting. In particular, the term connected as used herein is not meant to be limited to a direct connection, and allows for intermediary devices or components without limitation.

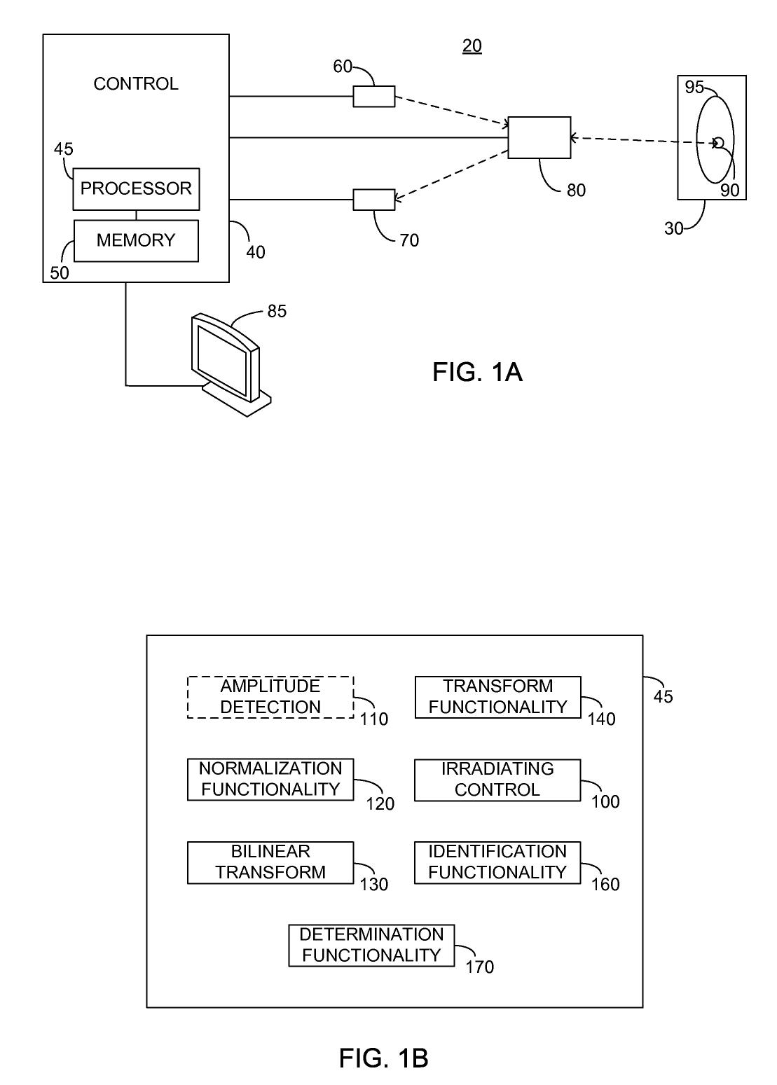

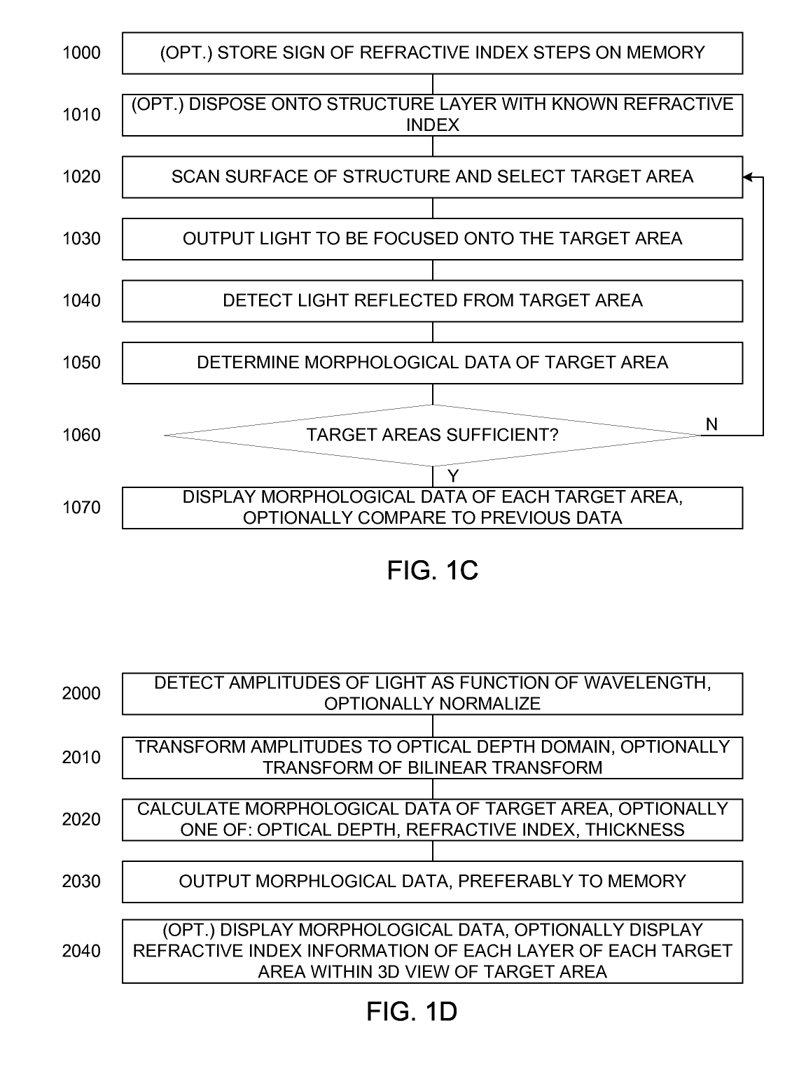

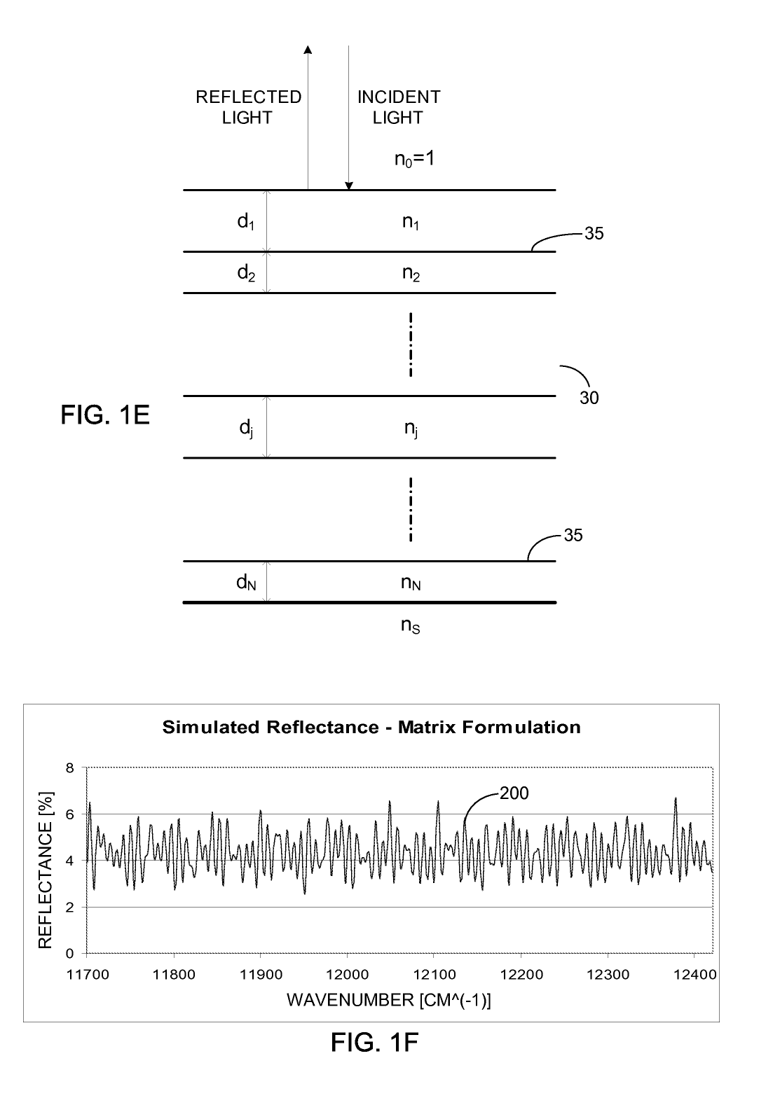

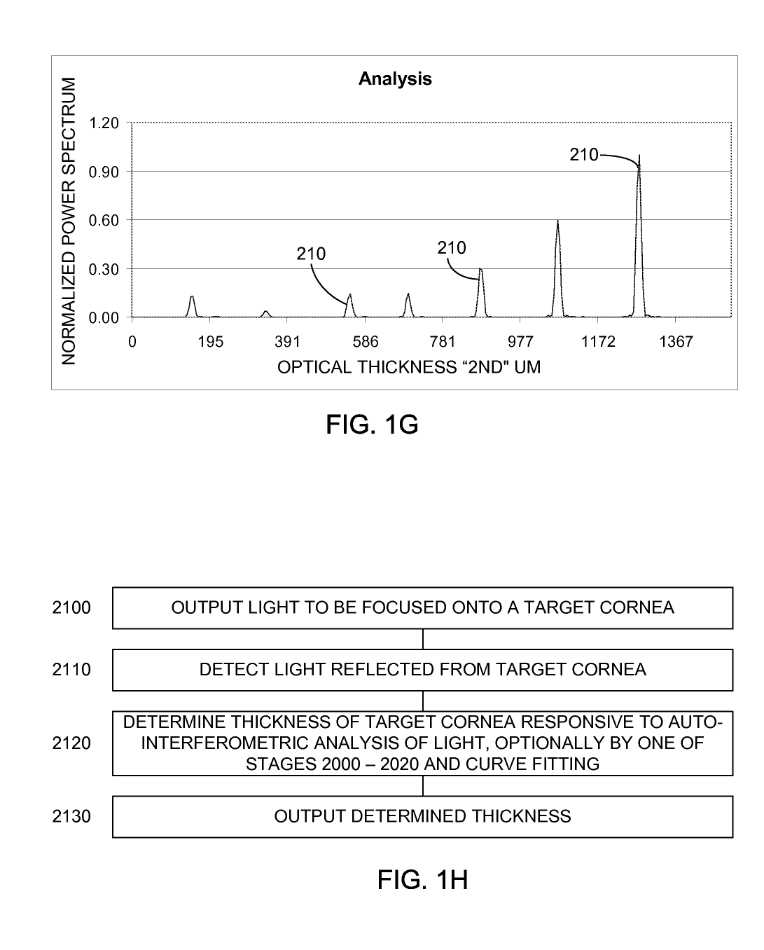

FIG. 1A illustrates a high level schematic diagram of an apparatus 20 arranged to determine morphological information of a structure 30 responsive to light reflected off structure 30. Apparatus 20 comprises: a control unit 40, comprising a processor 45 and a memory 50; a light source 60; a light detector 70; a target focusing functionality 80; and a display 85. Each of light source 60, light detector 70 and target focusing functionality 80 is in one embodiment in communication with control unit 40. Control unit 40 may be implemented in dedicated circuitry, or by a general purpose computing platform arranged to perform computer readable instructions read from a non-transitory storage device without limitation. In another embodiment, one or more of light source 60, light detector 70 and target focusing functionality 80 is controlled by an internal control unit.

In one embodiment, light source 60 outputs a broad band light, and in another embodiment light source 60 is a tunable light source responsive to control unit 40, particularly the wavelength of the light output by light source 60 is in such an embodiment responsive to an output of control unit 40. In one embodiment, light source 60 comprises a plurality of super luminescent diodes (SLDs). A plethora of such light sources 60 are commercially available, such as: the SLD-MS-series commercially available from Superlum of County Cork, Ireland; any of a plurality of ultra wideband light source modules commercially available from INPHENIX of Livermore, Calif.; and a Super Luminescent Light Emitting Diode Device commercially available from INPHENIX of Livermore, Calif. In another embodiment, light source 60 comprises a Tungsten-Halogen lamp. In one non-limiting embodiment, light source 60 is arranged to output light exhibiting a wavelength range of 0.8-0.85 um. In another non-limiting embodiment, light source 60 is arranged to output light exhibiting a wavelength ranged centered about any of a plurality of wavelengths, such as: 1.3 um; 1.5 um; or 1.6 um, without limitation.

In one embodiment, light detector 70 comprises one or more of a diffraction grating and a charge-coupled device (CCD) array. In one non-limiting embodiment, light detector 70 comprises a spectrometer module. A plethora of such light detectors 70 are commercially available, such as: the HR4000 Spectrometer commercially available from Ocean Optics, Inc. of Dunnedin, Fla., which exhibits a 1200 grooves/mm grating and a 5 micron slit; the CCS100 Compact CCD Spectrometer commercially available from Thorlabs of Newton, N.J.; and a high resolution spectrometer such as the AvaSpec-ULS3648-USB2 High Resolution Fiber Optic Spectrometer, commercially available from Avantes Inc. of Broomfield, Colo. In another non-limiting embodiment, light detector 70 comprises any one, or a combination of, a plurality of detector types, such as: a Silicon (Si) based detector; an Indium Gallium Arsenide (InGaAs) based detector; and a Lead Selenide (PbSe) based detector. In one embodiment, light detector 70 is arranged to detect specular reflection of light sourced by light source 60 after interaction with structure 30 and in another embodiment light detector 70 is arranged to detect diffuse reflection of light sourced by light source 60 after interaction with structure 30. In another embodiment, light detector 70 is arranged to detect both specular and diffuse reflection of light sourced by light source 60 after interaction with structure 30, without limitation.

In one embodiment, target focusing functionality 80 comprises a plurality of motors arranged to translate target focusing functionality along three orthogonal axes responsive to control unit 40. In one non-limiting example, target focusing functionality 80 may be translated along the three orthogonal axes by a GVS002 Scanning Galvanometer Mirror System, commercially available from Thorlabs of Newton, N.J. In one embodiment, target focusing functionality 80 comprises optical lenses arranged to focus light detected from light source 60 and further arranged to collect light reflected from structure 30 so that it is transferred to light detector 70, as will be described below. In one embodiment, target focusing functionality 80 additionally includes isolation devices, such as optical circulators, that allow proper routing of incoming or reflected light to the proper direction. In one embodiment, light traveling between light source 60, focusing functionality 80, structure 30 and light detector 70, travels through free space. In another embodiment, light traveling between light source 60, focusing functionality 80, structure 30 and light detector 70, is guided through optical fibers.

FIG. 1B illustrates a high level block diagram of processor 45 comprising: an irradiating control functionality 100; an optional amplitude detection functionality 110; a normalization functionality 120; a bilinear transform functionality 130; a domain transform functionality 140; an identification functionality 160; and a determination functionality 170. Each of irradiating control functionality 100, optional amplitude detection functionality 110, normalization functionality 120, bilinear transform functionality 130, domain transform functionality 140, identification functionality 160 and determination functionality 170 are in one embodiment implemented as automated processes within processor 45 of control unit 40, instructions for the automated processes stored on memory 50 in a machine readable format, preferably on a computer readable medium of fixed form, which may be a local storage drive, or may be a remote storage drive accessed over a network connection. Alternatively, dedicated hardware may be provided for each, or some, of irradiating control functionality 100, optional amplitude detection functionality 110, normalization functionality 120, bilinear transform functionality 130, domain transform functionality 140, identification functionality 160 and determination functionality 170 without exceeding the scope.

In an exemplary embodiment, light exiting light source 60 impacts structure 30, via target focusing functionality 80, at a near normal incidence, i.e. at about 90°+/−5% from a plane defined by the surface of structure 30. Light detector 70 is secured so as to detect light sourced by light source 60 reflected from structure 30 at a near normal incidence. In a non-limiting example light source 60 and light detector 70 are provided as a single controllable optical block. In one non-limiting embodiment light detector 70 comprises a lens. In one embodiment target focusing functionality 80 comprises a scanning galvanometer mirror system, such as the GVS002 commercially available from Thorlabs of Newton, N.J., as described above. Target focusing functionality 80 is arranged to scan the surface of structure 30 and further comprises a lens arranged to focus a beam of light detected from light source 60 onto a target area 90 of structure 30. In one embodiment the lens, or an additional lens, is also used to collect light reflected from structure 30 and transfer it to light detector 70, as will be described below. In one optional embodiment (not shown), light source 60 and light detector 70 are placed within control unit 40 and are in optical communication with structure 30 via fiber optics. As indicated above, in one non-limiting embodiment, light source 60 is constituted of a tunable laser light, in another embodiment light source 60 is constituted of a narrow bandwidth light source and in another embodiment light source 60 is constituted of a broad range light source, such as a white light or a halogen lamp. Light detector 70 may be constituted of a light detector with a light filter, a light detector with a tunable light filter, or a simple detector without exceeding the scope.

In operation, as will be described further below, in one embodiment a structure 30, such as the multi-layer structure illustrated in FIG. 1E, is scanned at a plurality of target areas 90. In another embodiment, a single target area 90 is scanned, as will be described below. Multi-layer structure 30 is comprised of a plurality of layers 35 numbered consecutively for identification from the external surface layer and proceeding away from the surface layer. For clarity, the surface layer is defined as the layer upon which light exiting light source 60 is first detected. In one non-limiting embodiment, each target area 90 exhibits a diameter of about 20 micrometers, the distance between the centers of adjacent target areas 90 is in one non-limiting embodiment about 10 micrometers. In one non-limiting embodiment, the plurality of target areas 90 forms an analysis area 95 of about 1 cm².

The method of analyzing an analysis area 95 of a multi-layer structure 30 is described in the high level flow chart of FIG. 1C and is implemented by apparatus 20. In optional stage 1000, if the sign of the refractive index steps for each of the interfaces of layers 35 is known, the information is stored on memory 50. In optional stage 1010, in the event that the refractive indices of all layers 35 are unknown, an additional layer (not shown) with a know refractive index is added to structure 30, preferably disposed on the surface thereof, i.e. on the surface of layer 1 thereof. In the embodiment where structure 30 is a cornea, the additional layer is in one embodiment liquid drops exhibiting a known refractive index. In stage 1020, control unit 40, via irradiating control functionality 100, controls target focusing functionality 80 to scan the surface of structure 30 and select a target area 90.

In stage 1030, control unit 40, in cooperation with irradiating control functionality 100, controls light source 60 to output light. In one embodiment, as will be described below, control unit 40 is arranged to disable the output of light from light source 60 after light detector 70 detects the reflected light from target area 90. In such an embodiment, control unit 40 is arranged to control light source 60 to initiate output of light for each analyzing of a target area 90. In another embodiment, light source 60 is disabled only after all of the plurality of target areas 90 have been analyzed. The beam of light is focused by target focusing functionality 80 onto the selected target area 90 of stage 1020 responsive to control unit 40. The diameter of the beam of light focused by target focusing functionality 80 is arranged such that the beam of light preferably covers the entirety of selected target area 90. In stage 1040, at least a portion of the light of stage 1030 is reflected from selected target area 90 and detected at light detector 70. In one embodiment, the reflected light is focused by target focusing functionality 80 onto light detector 70.

In stage 1050, morphological data of selected target area 90 is determined, as will be described in stages 2000-2030 of FIG. 1D. In stage 1060, control unit 40 determines if all desired target areas 90 were scanned. In one embodiment, the desired target areas 90 are predetermined and stored on memory 50. In the event that it is determined that all of the target areas 90 have not yet been scanned, stage 1020 as described above is performed. In the event that it is determined that all of the target areas 90 of interest have been scanned, or in the event that only a single target area 90 is scanned, in stage 1070 control unit 40 is arranged to display the determined morphological data of each target area 90, as will be described further below, on display 85. In one embodiment, the determined morphological data is transmitted by control unit 40 to an external computer via a local area network, wide area network, or the Internet, without limitation. In one embodiment, control unit 40 is arranged to display a 3 dimensional gray scale image of structure 30, as will be described further below. In the event that structure 30 is a biological structure, such as a cornea, in one embodiment the determined morphological data is compared to previously stored morphological data from an earlier analyzation session. The comparison may provide an indication as to whether there has been an improvement or deterioration in structure 30.

FIG. 1D illustrates a high level flow chart of the operation of processor 45 to determine morphological data as described above in stage 1050. In stage 2000, in one embodiment, control unit 40 steps the wavelength of light output from light source 60 by discrete even intervals, and further determines, responsive to optional amplitude detection functionality 110, the amplitude of the reflected light for each discrete wavelength. It is to be understood that stepping of the wavelength in discrete intervals is not meant to be limiting in any way, and sweeping of the wavelength may be performed, with samples taken at discrete intervals without exceeding the scope. In yet another embodiment, light source 60 continuously emits light in all desired spectra simultaneously. Light detector 70 may use an internal grating, prism or other tuning means in order to perform the spectrometric conversion so as to associate an amplitude with each discrete wavelength. In one non-limiting embodiment, light detector 70 is arranged to provide 2048 readings over the desired reflectance spectrum. In another embodiment, optional amplitude detection functionality 110 is integrated within light detector 70, such that light detector 70 provides control unit 40 with the amplitude of the reflected light as a function of wavelength.

As described above, control unit 40 detects the amplitude of the detected light as a function of the wavelength of the light output by light source 60. In one embodiment, control unit 40, responsive to normalization functionality 120, is further arranged to normalize the detected amplitude and preferably convert the measurements from wavelength to wave number for ease of calculation. The term wave number as utilized herein is defined as reciprocal of the wavelength, and is commonly used in spectroscopy, however this is not meant to be limiting in any way, and wavelength or frequency may be substituted, with the appropriate mathematical compensation, whenever the term wave number is utilized. Normalization is preferably performed based on the reflectance results measured for a known material with a known reflectance performance, such as aluminum, which has a reflectance of about 95%. A normalized value of the detected amplitude is thus determined.

In stage 2010, control unit 40 is arranged to transform the detected amplitudes as a function of wave number to the optical depth domain, preferably by performing a domain transform, such as a Fourier transform, of a bilinear transform of the detected amplitudes. Specifically, control unit 40, responsive to bilinear transform functionality 130, is further arranged to perform a bilinear transform on the detected amplitudes as a function of wave number, with the term bilinear transform as a function of wave number denoted B[R(w)], preferably defined as:

$$B [ R ( w ) ] = \frac{1 + R ( w )}{1 - R ( w )} \qquad EQ . 1$$

where R(w) is defined as the reflectance amplitude as a function of wave number w. In one embodiment, the amplitudes are determined as a fraction of light output reflected. B[R(w)] is illustrated in FIG. 1F as graph 200.

For an inhomogeneous structure, EQ. 1 can be expressed as:

$$B [ R ( w ) ] = {B}_{0} - {B}_{1} {\int }_{0}^{∞} \frac{1}{n ( x )} \frac{d n ( x )}{d x} cos [ 4 π w {\int }_{0}^{∞} n ( ξ ) d ξ ] d x \qquad EQ . 2$$

where n(x) is the refractive index at depth x, w is the wave number, B₀ is given as:

$${B}_{0} = \frac{{n}_{1}^{- 1} + {n}_{1}}{2} \qquad EQ . 3$$

and B₁ is given as:

$${B}_{1} = \frac{{n}_{1}^{- 1} - {n}_{1}}{2} \qquad EQ . 4$$

where n₁ is the refractive index of the first layer of the structure, i.e. the layer at the surface of structure 30 as described above.

For a multi-layer structure with small refractive index steps between adjacent layers, EQ. 2 can be expressed as:

$$B [ R ( w ) ] = {B}_{0} + {B}_{1} {\sum }_{j = 1}^{N} ( \frac{{n}_{j + 1} - {n}_{j}}{{n}_{j}} ) cos ( 4 π {\sum }_{i = 1}^{j} {n}_{i} {d}_{i} w ) \qquad EQ . 5$$

where N is the number of layers exhibiting a respective thickness dᵢ of the multi-layer structure.

Control unit 40, and particularly processor 45, is further arranged, responsive to domain transform functionality 140, to transform the bilinear transformed reflectance amplitudes to the optical thickness domain, preferably by performing a Fourier transform, even further preferably by performing a fast Fourier transform. There is no limitation to the domain transform, and autocorrelation or covariance methods may be used to determine optical thickness and amplitude relationships without limitation. In an exemplary embodiment a Fourier transform is performed by domain transform functionality 140, wherein the data is interpolated at equi-spaced wave-number points, high-pass filtered, windowed, zero padded to a specific number of points and a fast Fourier transform (FFT) algorithm is applied. From EQ. 5 it is evident that the Fourier analysis of B(w) leads to a spectrum in the optical thickness domain. The term optical thickness is defined as two times the refractive index times the thickness, denoted “2nd”, wherein “n” denotes the refractive index and “d” denotes the thickness of the layer, with the factor of 2 added to take into account that light must pass through the layer in both directions for reflectance data.

The bilinear transformed reflectance amplitudes transformed to the optical thickness domain exhibit a plurality of peaks 210, as illustrated in FIG. 1G. Control unit 40, and in particular processor 45, in cooperation with identification functionality 160, is preferably further arranged to identify the peaks from the bilinear transformed reflectance amplitudes transformed to the optical thickness domain. The amplitude of each peak is preferably given as:

$$A = {B}_{1} \frac{{n}_{j + 1} - {n}_{j}}{{n}_{j}} \qquad EQ . 6$$

In stage 2020, control unit 40 is in one embodiment further arranged, in cooperation with determination functionality 170, to determine morphological information of target area 90 responsive to identified peaks 210. In one embodiment, the optical thickness of at least one layer of target area 90 is determined. In another embodiment the refractive index of at least one layer is determined. Specifically, the refractive indices are determined responsive to the known, or determined, refractive index of the first layer. In the event that optional stage 1010, as described above, was performed and a first layer was externally added to structure 30, the refractive index of the first layer, i.e. n₁, is known. In the event that optional stage 1010 was not performed and the refractive indexes of all layers are unknown, the refractive index of layer n₁ is preferably determined responsive to the bilinear transform of EQ. 5. Specifically, as described above in relation to EQ. 1, B(w) is a function of the detected light amplitudes of stage 2010. The average of B(w) is determined responsive to the detected light amplitudes. From EQ. 5 it is clear that the average of B(w) is equal to B₀. From EQ. 3, n₁ is thus determined. The refractive indices of the subsequent layers are then determined responsive to the amplitudes of peaks 210 according to EQs. 4 and 6. The thickness of each layer can then be determined by dividing the respective peak 210 by twice the refractive index of the layer.

In one embodiment, the refractive index steps between each layer is determined and stored on memory 50. In the event structure 30 contains only a single layer, structure 30 can be considered as a multi-layer structure with the thickness of each additional layer being zero.

In one embodiment, in the event the average refractive index of structure 30 is known, the optical depth domain (2nd) spectrum of FIG. 1G is divided by 2*nₐᵥ, i.e. twice the average refractive index. The resultant spectrum is in the depth domain, i.e. is a function of the depth of structure 30. The amplitude of the spectrum at any specific depth may be presented as a gray scale representation of the 3 dimensional coordinate, i.e. the specific depth at the specific target area 90.

In stage 2030, the determined morphological information is output from determination functionality 170 and preferably stored on memory 50 and/or output on display 85. In stage 2040, as described above in relation to stage 1050, after all target areas 90 are analyzed, control unit 40 is in one embodiment arranged to display the stored morphological data on display 85. In one embodiment, where the refractive indices of the layers of structure 30 are determined for each target area 90, refractive index information of each layer 35 of each target area 90 is displayed on display 85 as a gray scale representation of a 3 dimensional view of target area 90. In one embodiment, the displayed refractive index information is the refractive index step between each layer 35 of each target area 90 and the refractive index steps within each layer 35 between adjacent target areas 90.

Advantageously, no interference is necessary to determine the morphological information responsive to the light reflected off of target area 90 and thus no reference mirrors are needed, reducing cost and complexity of apparatus 20 when compared to prior art OCT devices.

### Analysis of Simulated Reflectance Data

A simulated example of the use of the above method on a structure consisting of 7 layers disposed on a substrate is given herein as a non-limiting example. Table 1 represents the refractive indices, the depth and the optical depth of each layer:

TABLE I

| | Layer # | | | | | | | |
|---|---|---|---|---|---|---|---|---|
| | 1 | 2 | 3 | 4 | 5 | 6 | 7 | Substrate |
| Refractive Index | 1.52 | 1.5 | 1.49 | 1.47 | 1.45 | 1.42 | 1.38 | 1.33 |
| Thickness [μm] | 49.50 | 62.00 | 71.00 | 50.00 | 63.00 | 68.00 | 74.00 | |
| Optical Depth [μm] | 150.48 | 336.48 | 548.06 | 695.06 | 877.76 | 1070.83 | 1275.12 | |

A typical wavelength range of 0.8<λ<0.85 μm was assumed for the simulation. The reflectance data was interpolated at equi-spaced wave-number points, as illustrated in graph 200 of FIG. 1F. The Bilinear Transformation of Eq. 2 was applied and the result was high-pass filtered, windowed, zero padded to a specific number of points and a Fast Fourier Transform (FFT) was performed. The resulting spectrum is illustrated in FIG. 1G as described above.

Observed peaks 210 are representative of the seven interfaces between layers. The optical depths of such peaks are indicated in Table 1. If n₁ or n_{S}, i.e. the refractive index of the substrate, is known and there is structural information in regards of which indices are higher or lower at each interface, i.e. the sign of the refractive index steps at each interface are know, then all refractive indices and thicknesses can be determined, as described above.

For example, by averaging the bilinear transformed reflectance of EQ. 1 and responsive to EQ. 5, as described above, it is determined that n₁=1.52. The leftmost peak 210 occurs at an optical depth θ₁=151 μm and its amplitude is 0.0056. Therefore, the thickness of the first layer is d₁=151 μm/(2*1.52)=49.5 μm. According to EQ. 4, B₁=0.43 and according to EQ. 5, (n₂−n₁)=0.0056*1.52/0.43=0.02. Since n₁ is known to be 1.52, n₂=1.52−0.02=1.5. The thickness of the second layer is thus determined to be d₂=(θ2−θ1)/(2*1.5)=(337 μm−151 μm)/3=62 μm. This procedure is followed to determine the thickness of each of the various layers.

The minimal thickness of each layer which can be determined is given as:

$${d}_{MI N} = \frac{{λ}_{MA X} \cdot {λ}_{MI N}}{2 \cdot n \cdot ( {λ}_{M AX} - {λ}_{MI N} )} \qquad EQ . 7$$

where λ_{MAX} is the maximum wavelength of light detected by light detector 70, λ_{MIN} is the minimum wavelength of light detected by light detector 70 and n is the refractive index of the respective layer. For example, if n˜1.5 and as described above the wavelength range is 0.8<λ<0.85 μm, d_{MIN} is about 4.5 μm.

The maximal depth of structure 30 which can be determined is given as:

$${D}_{M AX} = \frac{{λ}_{MI N}^{2}}{4 \cdot {n}_{av} \cdot Δ λ} \qquad EQ . 8$$

where nₐᵥ is the average refractive index of the structure and Δλ is the wavelength resolution of light detector 70. For example, if nₐᵥ−1.5, Δλ=50 pm and λ_{MIN}=0.8 μm, the maximum depth which can be determined is approximately 2.1 mm.

FIG. 1H illustrates a high level flow chart of a method of measuring the thickness of a Cornea, according to certain embodiments. Today complex measurement arrangements, such as Orbscan II, commercially available from Bausch & Lomb of Aliso Viejo, Calif., are used for accurate analysis of the cornea before and after conducting LASIK (laser-assisted in situ keratomileusis) surgery. In addition to the full featured measurement suits, doctors use a simpler, single point cornea thickness measurement device. Each of these device types typically use an ultrasound measurement technique in order to measure the Cornea thickness, known also as “Ultrasound Pachymetry”. For example, the Kerasonix KSX-1000 ultrasound pachymeter, commercially available from DGH Technology of Exton, Pa., is used to measure the Cornea thickness. In the course of this measurement the doctor needs to apply anesthesia drops to the patient's eye. After waiting for a short period the doctor touches the approximate center of the cornea (where typically the Cornea reaches its minimal thickness) and the measurement is taken. The current ultrasound instruments are relatively simple and easy to use, but they involve application of drops and physical contact of the measuring tip with the patient's cornea. This involves some discomfort to the patient and the need to carefully sterilize the measurement tip in order to avoid contamination from one patient to another.

Apparatus 20 is in one embodiment utilized to perform Cornea thickness measurement. In stage 2100, target focusing functionality 80 is used to focus the beam of light on one target area 90, as described above in relation to stage 1030, preferably located immediately in front of the measurement device. In stage 2110, the reflected light is detected by light detector 70, as described above in relation to stage 1040. In stage 2120, the Cornea thickness measurement is then performed, responsive to a reflectometric analysis of the detected light of stage 2110. The term reflectometric analysis as used herein, refers to analysis of the detected light responsive only to interference of the light with itself at target area 90. As described above, apparatus 20 does not utilize reference mirrors in the subject analysis and interference from a reference signal is not provided for the reflected light from target area 90. Preferably, the reflectometric analysis is performed according to one of two methods. In a first method, morphological data of the target Cornea is determined, as described above in relation to stage 2000-2020. As described above, the morphological information of each layer of the target Cornea is determined as a function of the thickness of the particular layer. In particular, as described above, the thickness of each layer can be determined by dividing the respective peak 210 in the optical thickness domain by twice the refractive index of the layer. In a second method, the thickness of each layer of the cornea is determined by the detected light at light detector 70 and responsive to a curve fitting technique.

In stage 2130, the determined thickness of stage 2120 is output and preferably stored on memory 50 and/or output on display 85. In one embodiment, display 85 comprises a small screen facing the doctor and in another embodiment a speaker is further provided and the determined Cornea thickness is communicated by a voice-like output generated by control unit 40 and the provided speaker. This device can be very useful for initial screening of potential patients of LASIK surgery. It can also be very helpful in the diagnosis of Glaucoma which is manifested by Cornea thickness reduction from a typical value of about 500 nm to a value of about 400 nm.

The above operation allows Pachymetry to be performed by apparatus 20 which is smaller and consumes less power than traditional Pachymeters and therefore the entire device can be packaged in a portable device. In one embodiment, the portable device is pistol shaped where the doctor aims the frontal narrow “barrel” at the patient's approximate center of Cornea.

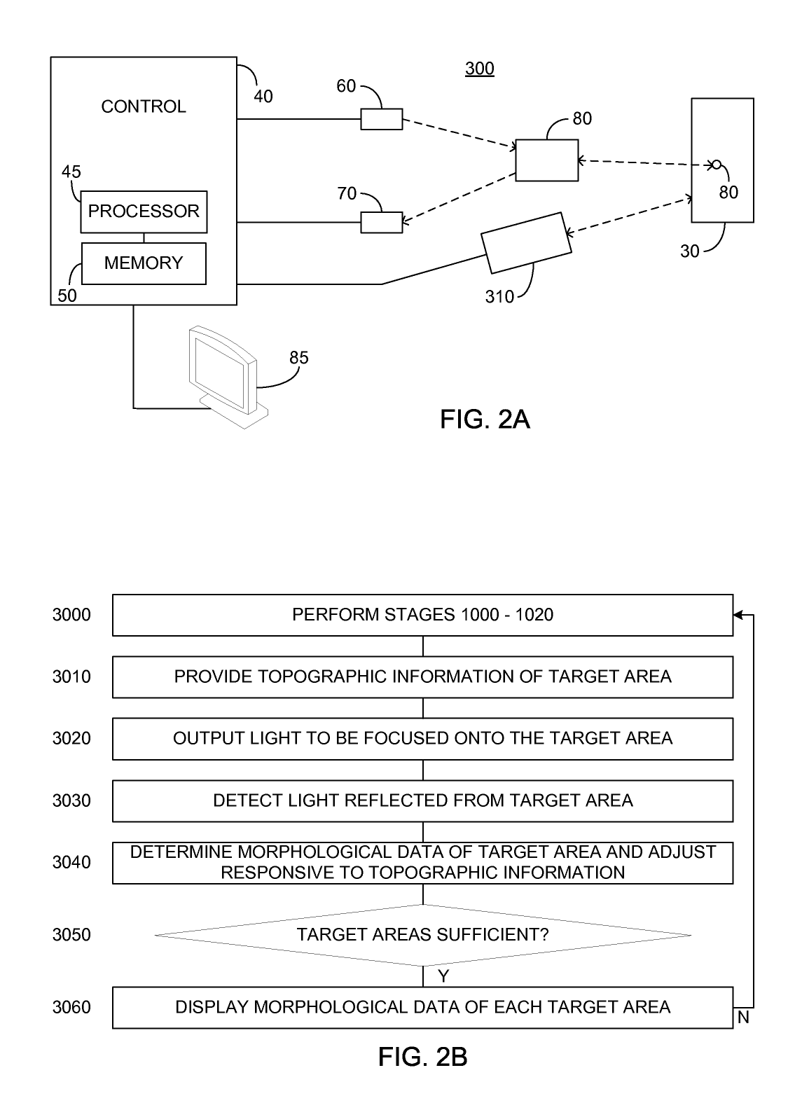

FIG. 2A illustrates a high level schematic diagram of an apparatus 300 arranged to determine morphological information of a structure 30 responsive to light reflected off structure 30. The construction and arrangement of apparatus 300 is in all respects similar to the construction and arrangement of apparatus 20 of FIGS. 1A-1B, with the addition of a surface topography functionality 310 in communication with control unit 40 and arranged to provide topographic information of the surface of structure 30. FIG. 2B illustrates a high level flow chart of the operation of apparatus 300, FIGS. 2A-2B being described together.

In operation, in stage 3000, stages 1000-1020 as described above are performed thereby selecting a target area 90 for analyses. In stage 3010, surface topography functionality 310 is arranged to scan a selected target area 90 and provide topographic information of the surface of selected target area 90. Preferably, surface topography functionality 310 defines a reference plane and determines the distance of the surface of selected target area 90 from the reference plane. Preferably, the defined reference plane is identical for all selected target areas 90. In one embodiment, the topographic information is stored on memory 50.

In stage 3020, control unit 40 controls light source 60 to output light, as described above in relation to stage 1030. In stage 3030, the light output by light source 60 is reflected from selected target area 90 and detected at light detector 70. In one embodiment, the reflected light is focused by target focusing functionality 80 onto light detector 70, as described above in relation to stage 1040.

In stage 3040, morphological information of selected target area 90 is determined, as described above in relation to stage 1050 and stages 2000-2030. In the event that the depth of selected target area 90 was determined, control unit 40 is further arranged to adjust the determined depth responsive to the topographic information of stage 3010. Specifically, the depth is adjusted responsive to the determined distance between the reference plane and the surface of selected target area 90. The adjusted depth is preferably stored on memory 50, as described above in relation stage 2030.

In stage 3050, as described above in relation stage 1060, control unit 40 determines if all desired target areas 90 were scanned. In one embodiment, the desired target areas 90 are predetermined and stored on memory 50. In the event that it is determined that all of the target areas 90 have not yet been scanned, stage 3020 as described above is performed. In the event that it is determined that no more target areas 90 need to be analyzed, in stage 3060, control unit 40 is arranged to display the determined morphological data of each target area 90, stored on memory 50, on display 85.

In another embodiment, surface topography functionality 310 is arranged to scan the surface of structure 30 and provide topographic information of the entirety, or a portion, of the surface of structure 30 which is then preferably stored on memory 50. Prior to display of the morphological information of stage 3060, control unit 40 is arranged to adjust the determined depths of each target area 90 responsive to the topographic information, as described above.

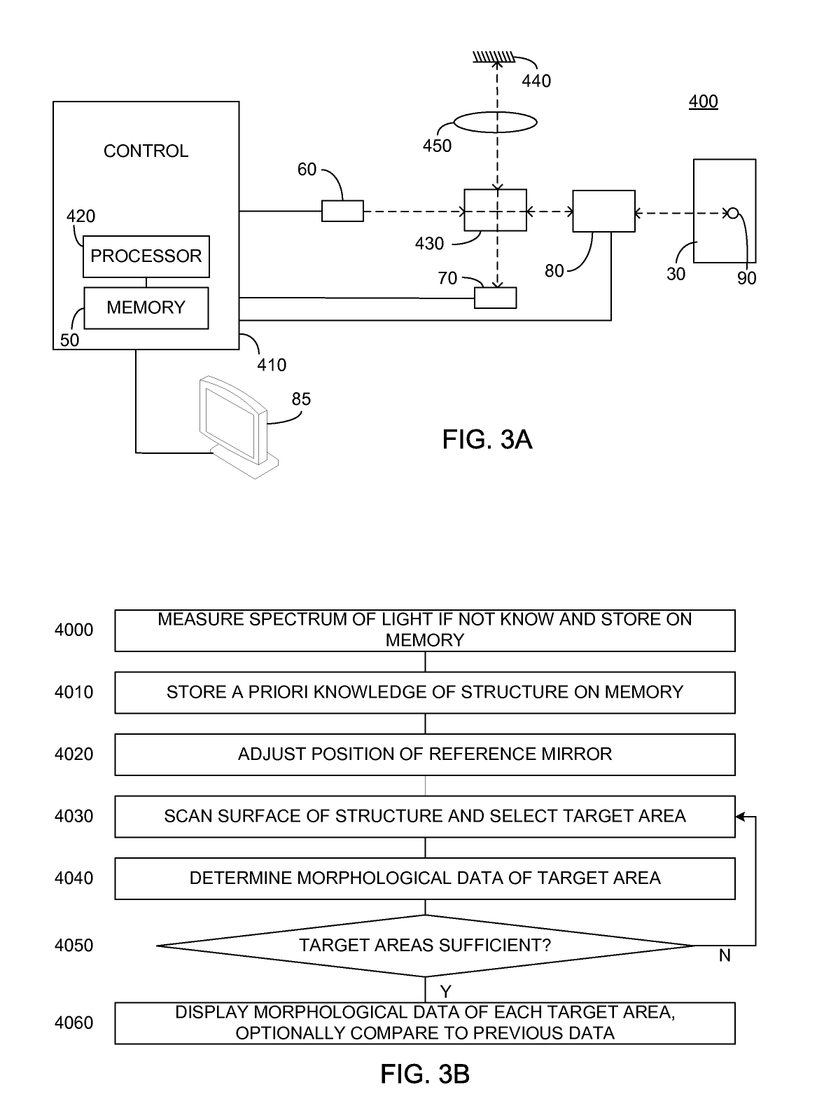

FIG. 3A illustrates a high level schematic diagram of an apparatus 400 arranged to determine morphological information of a structure 30 responsive to interference between light reflected off the structure and a reference light, both provided by a single light source 60. Apparatus 400 comprises: a control unit 410, comprising a processor 420 and a memory 50; a light source 60; a light detector 70; a target focusing functionality 80; a display 85; a light beam splitter 430; a reference mirror 440; and a focusing lens 450.

Each of light source 60, light detector 70, target focusing functionality 80, display 85 and reference mirror 440 is in one embodiment in communication with control unit 410. In another embodiment, one or more of light source 60, light detector 70, target focusing functionality 80, display 85 and reference mirror 440 is controlled by an internal control unit. Light beam splitter 430 is arranged to split a beam of light detected from light source 60 into a pair of light beams, a first light beam being directed to a target area 90 of structure 30 via target focusing functionality 80 and the second light beam being directed to reference mirror 440 via focusing lens 450. Reference mirror 440 is arranged to reflect the second light beam to light detector 70 via light beam splitter 430, the reflected second light beam performs an interference with light reflected off of structure 30, as will be described below. Reference mirror 440 preferably comprises a displacement mechanism arranged to translate reference mirror 440 along an axis in order to adjust the depth where structure 30 is analyzed.

FIG. 3B illustrates a high level flow chart of the method of the operation of apparatus 400, FIGS. 3A-3B being described together. In stage 4000, in the event the spectrum of the light arranged to be output from light source 60 is known, the information is stored on memory 50. In the event the spectrum is not known, it is measured in one of two methods. In the first method, control unit 410 controls light source 60 to output light which is detected by light detector 70. Control unit 410 then analyzes the detected light and determines the spectrum thereof. In the second method, a structure exhibiting pre-determined high absorption properties (not shown), and exhibiting a pre-determined spectral absorption pattern, is positioned between light beam splitter 430 and reference mirror 440 and arranged to absorb light directed to reference mirror 440. Additionally a structure exhibiting high reflectance properties is positioned to detect light from light source 60 via light beam splitter 430, the light reflected therefrom being detected by light detector 70. Control unit 410 then determines the spectrum of the detected light.

In stage 4010, a-priori knowledge of structure 30, specifically the sign of the refractive index steps between layer interfaces and the refractive index of at least one layer of structure 30, is stored, preferably on memory 50. In the event the refractive index of all layers of structure 30 are unknown, an external layer with a known refractive index is added on top of structure 30, as described above in relation to stage 1010. In stage 4020, reference mirror 440 is positioned such that the desired depth of structure 30 is analyzed. Specifically, the position of reference mirror 440 causes the interference between the light beam reflected off of structure 30 and the light beam reflected off of reference mirror 440 to represent the morphological information of structure 30 at a specific depth. In stage 4030, a target area 90 is selected by target focusing functionality, as described above in relation to stage 1020.

In stage 4040, morphological data of the selected target area 90 of stage 4030 is determined. In one embodiment, processor 420 contains dedicated circuitry to determine the morphological information of selected target area 90. In another embodiment, processor 420 is a computer platform containing computer readable instructions for determining the morphological information of target area 90. Specifically, control unit 410 controls light source 60 to output light. As described above, the output light is split by light beam splitter 430. A first beam of light is directed towards reference mirror 440 and a second beam of light is directed towards target area 90. Both beams of light are reflected off of their respective targets and interfere with each other, the interference being detected at light detector 70.

The intensity of the interference signal detected at light detector 70, denoted I(w) is given as:

$$I ( w ) = {I}_{S} ( w ) + {I}_{R} ( w ) + 2 \sqrt{{I}_{S} ( w ) {I}_{R} ( w )} {\sum }_{j = 1}^{N} {α}_{j , j + 1} cos ( 4 π {wn}_{j} {x}_{j} ) \qquad EQ . 9$$

where w is the wave-number, λ is the wavelength, I_{S} is the intensity of the light beam reflected off target area 90, I_{R} is the intensity of the light beam reflected off reference mirror 440, nⱼ is the refractive index of the layer j; and α_{j,j+1} is the square root of the reflectivity of the sample “r_{j,j+1}” (at depth xⱼ, between layers j and j+1). Depth information of structure 30 is preferably determined by means of an inverse Fourier transform on the signal detected at light detector 70, the transformed signal being given as:

$${\vert {F}^{- 1} [ I ( w ) ] \vert}^{2} = {Γ}^{2} ( x ) ⊗ [ δ ( 0 ) + {\sum }_{j = 1}^{N} {r}_{j , j + 1} δ ( x - {x}_{j} ) + {\sum }_{j = 1}^{N} {r}_{j , j + 1} δ ( x + {x}_{j} ) + A ( {x}_{j} ) ] \qquad EQ . 10$$

where γ(x) is the envelope of the coherence function, and δ(0) and A(xⱼ) represent reference and sample autocorrelations, respectively. The second and third terms within the brackets of EQ. 10 are produced by the interference between the reflected light beams of target area 90 and reference mirror 440. Depth information is extracted responsive to the Dirac delta functions “δ(x−xⱼ)”, whose spectral positions represent the depth “xⱼ” of structural interfaces.

A first order approximation of the reflectivity of an inhomogeneous refractive index profile is given as:

$$r ( w ) = \frac{{r}_{01} - \frac{1}{2} {\int }_{0}^{∞} \frac{1}{n ( x )} \frac{d n ( x )}{d x} {e}^{- i 4 π w {\int }_{0}^{x} n ( ξ ) d ξ} d x}{1 - \frac{{r}_{01}}{2} {\int }_{0}^{∞} \frac{1}{n ( x )} \frac{d n ( x )}{d x} {e}^{- i 4 π w {\int }_{0}^{x} n ( ξ ) d ξ} d x} \qquad EQ . 11$$

where r₀₁ is the reflectivity between air and the surface of the sample. When dealing with very small refractive index changes, for example those observed in retinal analysis, EQ. 11 can be approximated to:

$$r ( w ) \approx {r}_{01} - \frac{1}{2} {\int }_{0}^{∞} \frac{1}{n ( x )} \frac{d n ( x )}{d x} {e}^{- i 4 π w {\int }_{0}^{x} n ( ξ ) d ξ} d x \qquad EQ . 12$$

Solving EQ. 12 for a multi-layer structure with small refractive index steps between layers, EQ. 12 becomes:

$$r ( w ) \approx {r}_{01} - \frac{1}{2} {\sum }_{j = 1}^{N} \frac{{n}_{j + 1} - {n}_{j}}{{n}_{j}} {e}^{- i 4 π w {\sum }_{i = 1}^{j} {n}_{i} {d}_{i}} \qquad EQ . 13$$

EQ. 13 shows that “r(w)”, i.e. the reflectivity expressed as a function of the wave-number, possesses a spectrum in the optical thickness domain “2nd”. The positions of the spectrum peaks correspond to interfaces between layers and the peak amplitudes are proportional to the refractive index steps at the respective interfaces.

By taking into account the reflectivity of EQ. 13, EQs. 9 and 10 can be given a more appropriate expression, as functions of the optical depth “θj”, such that EQ. 9 is given as:

$$I ( w ) = {I}_{S} ( w ) + {I}_{R} ( w ) + 2 \sqrt{{I}_{S} ( w ) {I}_{R} ( w )} {\sum }_{j = 1}^{N} {α}_{j , j + 1} cos ( 2 {πθ}_{j} w ) \qquad EQ . 14$$

and EQ. 10 is given as:

$${\vert {F}^{- 1} [ I ( w ) ] \vert}^{2} = {Γ}^{2} ( θ ) ⊗ [ \begin{matrix}δ ( 0 ) + \frac{1}{2} {\sum }_{j = 1}^{N} \frac{Δ {n}_{j}}{{n}_{j}} δ ( θ - {θ}_{j} ) + \\ \frac{1}{2} {\sum }_{j = 1}^{N} \frac{Δ {n}_{j}}{{n}_{j}} δ ( θ + {θ}_{j} ) + A ( {θ}_{j} )\end{matrix} ] \qquad EQ . 15$$

where θj is given as:

$${θ}_{j} = 2 {\sum }_{i = 1}^{j} {n}_{i} {d}_{i} \qquad EQ . 16$$

The refractive index step between layers is Δnⱼ=nⱼ₊₁−nⱼ. For small refractive index steps the Fresnel reflectivity coefficient between layers j and j+1 of EQ. 10 approaches:

$${r}_{j , j + 1} = - \frac{Δ {n}_{j}}{2 {n}_{j}} \qquad EQ . 17$$

EQ. 15 is expressed as:

$${\vert {F}^{- 1} [ I ( w ) ] \vert}^{2} = {Γ}^{2} ( θ ) ⊗ [ \begin{matrix}δ ( 0 ) + \frac{1}{2 {n}_{av}} {\sum }_{j = 1}^{N} Δ {n}_{j} δ ( θ - {θ}_{j} ) + \\ \frac{1}{2 {n}_{av}} {\sum }_{j = 1}^{N} Δ {n}_{j} δ ( θ + {θ}_{j} ) + A ( {θ}_{j} )\end{matrix} ] \qquad EQ . 18$$

In EQ. 18, an average value nₐᵥ replaces nⱼ in the denominator of the second and third terms within the brackets. Based on the assumption of small refractive index steps, such replacement introduces negligible error, and a value for nₐᵥ may be obtained from known statistical values. For example, refractive index values around 1.38 are often used for cornea analysis.

In order to measure the refractive index steps at the layer interfaces, compensation for the influence of light source 60 and normalization of the spectrum amplitudes according to a known standard are preferred.

Compensation for source effects is accomplished by dividing the detected interference signal of EQ. 14 by its known spectrum. As described above in relation to stage 4000, in the event that the spectrum of light from light source 60 is not known, it is measured. Alternatively, an inverse Fourier transform is performed on the detected light beam at light detector 70, which leads to Γ²(θ) of EQ. 15. Thus, when analyzing a structure 30, the amplitudes of the spectral peaks of interest (the second and third terms within the brackets of EQ. 15) are determined and divided by the correspondent Γ²(θ) associated values.

Normalization is achieved by performing the above analysis on a structure (not shown) exhibiting a known refractive index. Measuring the amplitude of the resultant inverse Fourier transform interface peak leads to a scaling factor for EQ. 18.

The refractive index steps between layers of structure 30 are calculated according to EQ. 17 or 18, i.e. the amplitude of the normalized spectral peak “j” is determined and multiplied by two times nₐᵥ, leading to Δnⱼ. Having calculated the refractive index steps between layers, the refractive indices can then be determined responsive to the known refractive index of one of the layers of structure 30 stored on memory 50 in stage 4010. The sign of Δnⱼ indicates if the index of refraction increases or decreases at the interface. As this is not known from the Inverse Fourier transformation, a-priori knowledge of the sign of the refractive index are preferably utilized, as described above in relation to stage 4010.

EQs. 14 and 16 reveal that the determination of the refractive index steps between interfaces improves thickness estimation. Thus, subsequent to the calculation of the refractive index steps, the thickness of each layer is preferably more accurately determined.

In stage 4050, control unit 410 determines if all desired target areas 90 were scanned. In one embodiment, the desired target areas 90 are predetermined and stored on memory 50. In the event that it is determined that that all of the target areas 90 have not yet been scanned, stage 4030 as described above is performed. In the event that it is determined that no more target areas 90 need to be analyzed, in stage 4060, control unit 410 is arranged to display the determined morphological data of each target area 90 stored on memory 50, as will be described further below, on display 85. In the event that structure 30 is a biological structure, such as a cornea, the determined morphological data is preferably compared to previously stored morphological data from an earlier analysis session, with the comparison output to display 85. The comparison can indicate whether there has been an improvement or deterioration in structure 30.

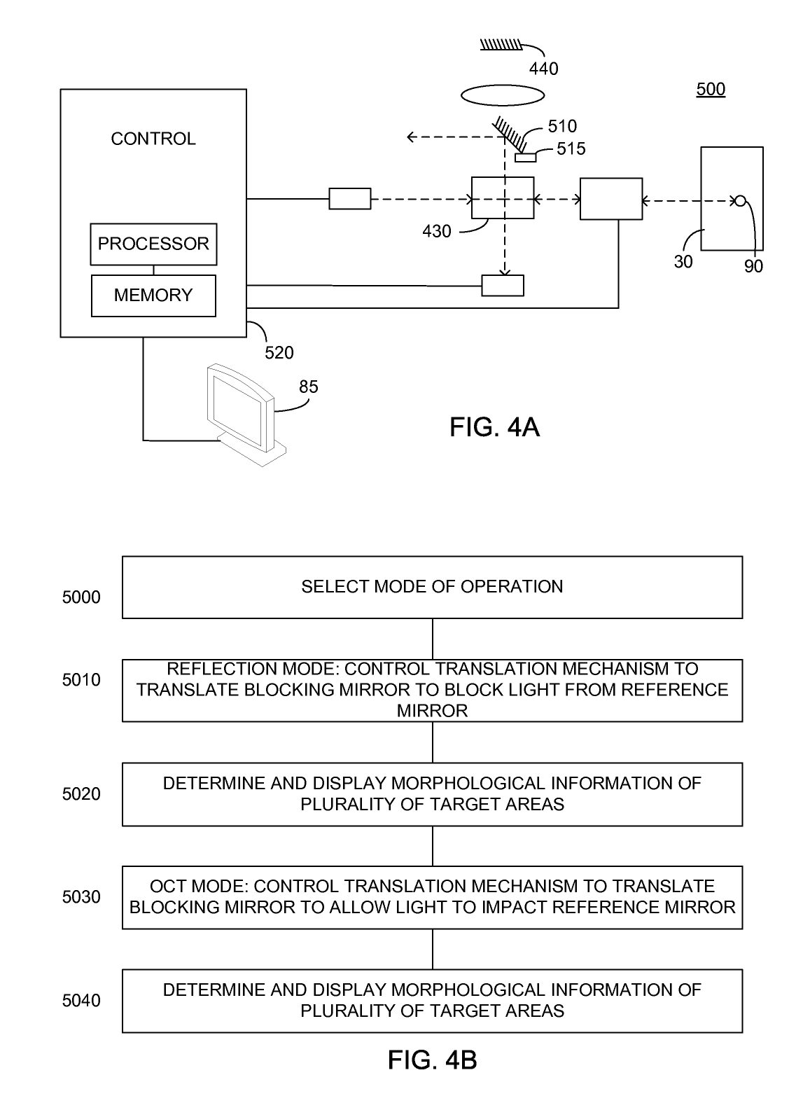

FIG. 4A illustrates a high level schematic diagram of an apparatus 500 arranged to determine morphological information of a structure 30 in a pair of modes. The construction and arrangement of apparatus 500 is in all respects similar to the construction and arrangement of apparatus 400 of FIG. 3A, with the exception that a blocking mirror 510 is provided in communication with a control unit 520 (communication path not shown). Blocking mirror 510 is in communication with a translation mechanism 515 arranged to alternately: in a reflectance mode translate the blocking mirror 510 to a position so as to block any light from light beam splitter 430 from reaching reference mirror 440; and in an optical coherence tomography mode translate the blocking mirror 510 to a position so as to allow a light beam from light beam splitter 430 to impact reference mirror 440, as described above in relation to FIGS. 3A-3B. In one embodiment (not shown), a plurality of blocking mirrors 510 are provided, at least one of the blocking mirrors 510 in communication with a respective translation mechanism 515. In one embodiment, control unit 520 comprises processor 45 of control unit 40 of FIG. 1B and processor 420 of FIG. 3A. FIG. 4B illustrates a high level flow chart of the method of operation of apparatus 500, FIGS. 4A-4B being described together.

In operation, in stage 5000, a mode of operation is selected. In one embodiment, the mode of operation is selected by a user via a user input device (not shown). In the event that the reflectance mode is selected, in stage 5010, control unit 520 controls translation mechanism 515 to translate blocking mirror 510 to a position so as to block light from light beam splitter 430 from reaching reference mirror 440. In one embodiment, blocking mirror 510 is arranged to direct light detected from light beam splitter 430 to a structure (not shown) exhibiting very high absorption properties. In another embodiment, blocking mirror 510 is arranged to direct light detected from light beam splitter 430 away from apparatus 500 such that the light beam doesn't interfere with the light beam directed towards structure 30, or the light reflected therefrom. In stage 5020, morphological information of a plurality of target areas 90 are determined and output, optionally by displaying the determined morphological information on display 85, as described above in relation to stages 1000-1050.

In the event that the OCT mode is selected, in stage 5030, control unit 520 controls translation mechanism 515 to translate blocking mirror 510 to a position so as to allow a light beam from light beam splitter 430 to impact reference mirror 440. In stage 5040, morphological information of a plurality of target areas 90 are determined and output, optionally by displaying the determined morphological information on display 85, as described above in relation to stages 3000-3040.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The terms “include”, “comprise” and “have” and their conjugates as used herein mean “including but not necessarily limited to”. The term “connected” is not limited to a direct connection, and connection via intermediary devices is specifically included.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. An apparatus arranged to analyze a structure, the apparatus comprising:
a control unit;
a target focusing functionality;
a light source in optical communication with said target focusing functionality, said light source arranged, in cooperation with said target focusing functionality, to irradiate a plurality of target areas of the structure, each target area comprising a particular area of at least the surface layer of the structure; and
a light detector in communication with said control unit, and arranged to detect said irradiated light from said light source after interaction with each target area,
said control unit arranged, for each of a plurality of target areas of the structure, to:
control said target focusing functionality such that the target area is irradiated by said light source and said light detector detects said irradiated light from said light source after interaction with the target area;
transform the amplitude of the detected light to the optical thickness domain, said transform comprising, for each wave number of the detected light, the equation (1+R)/(1−R), wherein R represents the amplitude of the detected light;
determine morphological information of the target area responsive to said performed transform; and
output said determined morphological information.

2. (canceled)

3. The apparatus of claim 1, wherein the structure comprises a plurality of layers, the apparatus further comprising:
a display, in communication with said control unit and arranged to display said output morphological information,
wherein each target area comprises a particular area of the plurality of layers of the structure,
wherein said morphological information comprises thickness information for at least one of the plurality of layers of each target area, and
wherein a three dimensional view of each target area is displayed on said display, the determined thickness information of each target area being displayed within the three dimensional view.

4. The apparatus of claim 1, wherein said morphological information comprises optical thickness information of a layer of each target area.

5. The apparatus of claim 1, wherein said morphological information comprises the refractive index of a layer of each target area.

6. The apparatus of claim 1, wherein said morphological information comprises the thickness of a layer of each target area.

7. The apparatus of claim 1, wherein said control unit is further arranged to receive topographic information of a surface of each target area and adjust said calculated morphological information responsive to said received topographic information.

8. The apparatus of claim 1, wherein the structure is a Cornea and said morphological information comprises the thickness of the Cornea.

9. A method of analysis of a structure, the method comprising, for each of a plurality of target areas of the structure:
irradiating the target area of the structure with a first beam of light;
detecting the amplitude of said first beam of light after interaction with the target area, the amplitude detected as a function of wavelength;
transforming said detected amplitude to the optical thickness domain, said transforming comprising, for each wave number of the detected light, performing the equation (1+R)/(1−R), wherein R represents said detected amplitude;
determining morphological information of the target area responsive to said transforming; and
outputting said determined morphological information,
wherein each target area comprises a particular area of at least the surface layer of the structure.

10. The method of claim 9, wherein each target area of the structure comprises a plurality of inhomogeneous layers.

11. The method of claim 9, wherein each target area comprises a particular area of a plurality of layers of the structure, said morphological information comprising thickness information regarding each of the plurality of layers of each target area, the method further comprising:
displaying a three dimensional view of each target area; and
displaying the determined thickness information of each layer of each target area within said displayed three dimensional view.

12. The method of claim 9, wherein said morphological information comprises optical thickness information of a layer of each target area.

13. The method of claim 9, wherein said morphological information comprises the refractive index of a layer of each target area.

14. The method of claim 9, wherein said morphological information comprises the thickness of a layer of each target area.

15. The method of claim 9, further comprising disposing a layer of optical liquid, exhibiting a known refractive index, on the structure.

16. The method of claim 9, further comprising:
receiving topographic information of a surface of each target area; and
adjusting said calculated morphological information responsive to said received topographic information.

17. The method of claim 9, wherein the structure is a Cornea and said morphological information comprises the thickness of the Cornea.

18. A control unit arranged to analyze light reflected off each of a plurality of target areas of a structure, each target area comprising a particular area of at least the surface layer of the structure, the amplitude of the light reflected off each target area detected by a detector arranged to output to said control unit information regarding the detected amplitude as a function of wavelength, the control unit comprising:
an irradiating control functionality arranged, for each of a plurality of target areas of the structure, to control a target focusing functionality such that the target area is irradiated by light;
a transform functionality arranged to transform the detected amplitude to the optical thickness domain, said transform comprising, for each wave number of the detected light, the equation (1+R)/(1−R), wherein R represents the amplitude of the detected light; and
a determining functionality arranged to determine morphological information of the target area responsive to said transform and further arranged to output said determined morphological information.

19. (canceled)

20. The control unit of claim 19, wherein the control unit is in communication with a display arranged to display said output morphological information,
wherein each target area comprises a particular area of a plurality of layers of the structure,
wherein said morphological information comprises thickness information for each of a plurality of layers of each target area, and
wherein said control unit is arranged to cause said display to display a three dimensional view of the target area and to display the determined refractive index information of each layer of each target area within the three dimensional view.

21. The control unit of claim 19, wherein said morphological information comprises one of:
optical thickness information of a layer of each target area;
the refractive index of a layer of each target area; and
the thickness of a layer of each target area.

22. (canceled)

23. (canceled)

24. The control unit of claim 19, wherein said determining functionality is further arranged to receive topographic information of a surface of each target area and adjust said calculated morphological information responsive to said received topographic information.

25-32. (canceled)

33. The control unit of claim 20, wherein the plurality of layers of each target area are inhomogeneous.
